(19) United States
(12) Patent Application Publication
(10) Pub. No.: US 2001/0005718 A1
(43) Pub. Date: Jun. 28, 2001

(54) Apparatus and process for rapid hybridization

(76) Inventors: Yang Wen-Tung, Taichung (TW); Lee Kan-Hung, Taipei (TW); Tsai Chuan-Mei, Hsinchu (TW); Shih Yu-Hau, ChanHwa (TW); Wang Yih-Weng, Tainan (TW)

Correspondence Address: SCULLY, SCOTT, MURPHY & PRESSER, 400 Garden City Plaza, Garden City, NY, 11530, US

(21) Appl. No.: 09/757,017

(22) Filed: Jan. 8, 2001

Related U.S. Application Data
(63) Continuation-in-part of application No. 09/348,604, filed on Jul. 6, 1999, now abandoned.

(30) Foreign Application Priority Data
Apr. 16, 1999 (TW) 88106121

(51) Int. Cl. A61K 31/70; A01N 43/04; C12M 1/34; C12M 3/00

(52) U.S. Cl. 514/44; 435/6; 435/287.2

(57) ABSTRACT

The invention provides an apparatus for rapid hybridization reaction which comprises a holder for loading a substrate and an electric field generator comprising a power controller and at least an electrode placed above or below the holder for generating a direction-convertible electric field at least to cover the substrate. The invention also provides a process for rapid hybridization reaction which comprises applying an upward electric field to hybridization substances, zeroing the electric field and changing direction of the electric field downward. The hybridization reaction rate increases and reaction time greatly reduces and meanwhile reaction specificity and opportunity equality are retained.

### BACKGROUND OF THE INVENTION

1. Field of the Invention

The present invention relates to a rapid hybridization reaction in the molecular biological field and, more specifically, relates to the apparatus and process for a rapid nucleic acid hybridization reaction.

2. Description of the Related Art

Nucleic acid hybridization analysis has been widely applied to sequencing nucleic acid, gene mutation analysis and clinical detection of bacterium and viruses. The hybridization is a reaction combining two single stranded nucleic acids to one double stranded nucleic acid. The hybridization is classified into solution/liquid hybridization as taught in P. Wattre, 55 Ann. Biol. Clin. 25-31, 1997 and solid hybridization referring to E. M. Southern 98 J. Mol. Biol. 503-517, 1975, F. C. Kafatos et al., 7 Nucleic Acids Res. 1541-1552, 1979, D. Nanibhushan and D. Rabin, EP0228075, Jul. 8, 1987, M. Grunstein and D. S. Hogness. 72 Proc. Nat. Acad. Sci. U.S.A., 3961-3965, 1975, A. R. Dunn and J. A. Hassell, 12 Cell 23-36, 1977, and U.S. Pat. No. 4,563,419 invented by Ranki et al. One of the marked shortcomings of the former one is: since two groups of probe and sample nucleic acids of single stranded forms are both in a solution, reactions of the same group are likely to occur and reactions of different groups are falsely reduced.

The solid hybridization avoids the above shortcomings by fixing one group of nucleic acid on a substrate. There are several forms of solid hybridization and they are all originated from Southern blot-hybridization procedure which was disclosed by E. M. Southern in 98 J. Mol. Biol. 503-517, 1975. The procedure lay in transferring DNA to a nitrocellulose membrane for complementary nucleic acid hybridization. Dot blot hybridization disclosed by F. C. Kafatos et al. in 7 Nucleic Acids Res. 1541-1552, 1979 dotted target DNA on a a membrane to conduct a hybridization reaction with a probe nucleic acid labeled with radioactive or other color development substances. The technology has been widely applied in gene mutation analysis as disclosed by D. Nanibhushan and D. Rabin in EP0228075 and other related researches in molecular biology.

Colony hybridization as taught by M. Grunstein and D. S. Hogness in 72 Proc. Nat. Acad. Sci. U.S.A., 3961-3965, 1975 fixed microorganisms such as bacteria, phage or other microorganisms on membrane and in situ broke the same to release and denature the nucleic acid thereof. The denatured nucleic acid was fixed on a membrane and reacted with nucleic acid labeled with radioactive or other color development substances.

Sandwich hybridization disclosed by A. R. Dunn and J. A. Hassell in 12 Cell 23-36, 1977 and by Ranki et al. in U.S. Pat. No. 4,563,419 utilized two groups of probe nucleic acids. The first group was fixed on a membrane and reacted with a target nucleic acid and then the second group reacted with the new combinations of the first group and the target nucleic acid.

The common shortcomings of the above hybridization systems are: first, nucleic acid molecules diffuse in Brownian movement towards nucleic acid molecules transferred on a membrane, i.e. a kind of passive hybridization, and the diffusion speed is slow and takes time, generally more than ten hours or overnight, to react; and second, nucleic acid in a solution renatures and falsely binds with a complementary single stranded nucleic acid of the same group to one double stranded ones in DNA, or forms secondary structures in RNA and the hybridization efficiency is poor.

The specificity and sensitivity are also important factors to be considered. Most nucleic acid molecules are highly complex and tend toward partial or mismatched hybridization which results in poor specificity. Meanwhile, nucleic acid molecules numbering less than 100,000 are generally not detectable since nucleic acids are in a solution and the sensitivity is poor. Furthermore, nucleic acid is highly affinitive to most substances and the free movement of nucleic acid in a solution results in the combination of a nucleic acid and a substrate and severe background noises.

Conventional methods increase the specificity of hybridization reaction by increasing reaction temperatures, adjusting ion concentrations in a solution, adjusting rinse conditions after hybridization, or adding substances such as urea or formamide to help isolate double stranded nucleic acids. Whereas the optimum reaction condition is hard to adjust to and sensitivity often reduces as specificity increases.

Some research focused on additive substances to increase reaction, such as polyethylene glycol disclosed by M. Renz et al. in 12, Nucleic Acids Res., 3435-3444, 1984, dextran sulfate disclosed by G. M. Wahl, et al. in 76, Proc. Nat. Acad. Sci. U.S.A., 3683-3687, 1979, polyacrylate or polymethacrylate disclosed by S. J. Boguslawski, and L. H. D. Anderson in U.S. Pat. No. 4,689,294. Nevertheless, the shortcomings mentioned above still exist.

In situ hybridization has been applied to detecting gene and chromosome abnormality in cells. The DNA chip by M. Barinaga in 253 Science, 1489, 1991 fixed nucleic acid sequences on a chip for hybridization. This technology confines dot blot, colony and sandwich hybridization to a microchip and locally concentrates nucleic acids. Though sensitivity is increased, the other shortcomings remain unresolved.

The other hybridization technology applies a point electric field to a substrate fixed with nucleic acid, referring to U.S. Pat. No. 5,605,662, U.S. Pat. No. 5,849,486 and U.S. Pat. No. 5,632,957 by Heller et al. Although the point electrode hybridization avoids low sensitivity and specificity of the conventional diffusion methods, the point electrode leads to uneven opportunity for hybridization and results in pseudo-positive hybridization. Furthermore, as disclosed by Heller et al., the electrodes of the electronic device are formed in the part of a printed circuit board. It shows that the substrate with the nucleic acids is in combination with the electrode of the circuited board. Therefore, the electrode with such substrate cannot be repeatedly used. That is, said electrode only can be used once in the hybridization reaction. The cheaper materials such as nylon membrane and nitrocellulose membrane customarily used in the hybridization reaction cannot be used in the device of Heller et al.

JP03047097 discloses a hybridization process and a method for detecting genetic variation employing same and an apparatus therefore. As disclosed in JP03047097, a gel solution comprising a DNA probe is used to obtain the electrophoretic carrier substrate. The DNA fragments are moved in the gel electrophoretic carrier substrate to hybridize with the DNA probe, which is fixed on the electrophoretic carrier substrate. Obviously, the substrate of JP03047097 cannot be repeatedly used. Moreover, such system of JP03047097 has the disadvantages as follows: (1) the 5′ end of a probe must be modified and then immobilized with acrylamide in order to perform the electrophoresis; (2) such system cannot use the substrate immobilized with probe which is customarily used in the art; and (3) the nucleic acid molecules cannot be fully hybridized with the probes because the nucleic acid molecules are merely moved with one same direction.

JP080154656 teaches an apparatus for a nucleic acid hybridization test provided with a substrate, an electroconductive layer and a power source for applying voltage to the electroconductive layer. The substrate plate of JP080154656 has an electroconductive layer on either the front or the back side, or both side. Therefore, such substrate cannot be detached. Moreover, as shown in the specification of JP080154656, the substrate is coated with the conductive materials such as chromium film and dielectric oxide to from an electrode. Thus, the cost of the apparatus of JP080154656 is increased.

An electric field has been utilized to speed up the reaction of antibody and antigen. Zhang et al. in CN1092174 disclosed an accelerator for enzyme-labeled immunosorbent assay (ELISA) wherein plastic plates are loaded with antigen and antibody and placed on copper plates which are connected to a high frequency vibrator. The vibrator transformed alternative current in 220 volts potential to a high frequency electric field which vibrated the antigen and antibody in a high speed and completed the reaction in several minutes. The high speed electric field could not be applied in hybridization since the hybridization of nucleic acids was a process for combinations of at least ten complementary alkalinic groups in two single strained nucleic acids. The hybridization reaction required a certain extention of time while high frequency vibration would disrupt the reaction. Furthermore, high frequency vibration neither mixed nucleic acids well nor concentrated the nucleic acids. The hybridization efficiency was low. The reaction specificity was low since high frequency vibration did not exclude mismatched hybridization.

I. Karube et al. in WO 9423287 taught the combinations of antigen and antibody by placing two electrodes adsorbed with known antigen and antibody in a solution and reacting the known antigen and antibody with antigen and antibody to be detected in the solution between the two electrodes. The antigen or antibody in the solution moved to the electrodes and reacted with the antigen or antibody on the electrodes to form antigen-antibody complex. The concentration could be known by detecting conductivity. The method moved antigen or antibody by electric field but did not control and speed the forming of complex. The method could not be applied in the hybridization of nucleic acids since it neither speeded up the reaction nor excluded non-specific hybridization. Furthermore, the electrodes should be place in a solution. The application was restricted.

In summary, the disadvantages of the prior art lie in that: first, hybridization speed and efficiency are poor by nucleic acids diffusion; the reaction takes more than ten hours and therefore the specificity and sensitivity are hard to control; and pseudo-negativity occurs when nucleic acid concentration is low while pseudo-positivist occurs under the occasion of partial complementary hybridization; second, a point electric field may speed up reaction locally but could not provide equal opportunity for hybridization; and third, a high frequency electric field may speed up the combination of antigen and antibody but does not provide sufficient time for the hybridization reaction of nucleic acids nor exclude non-specific hybridization.

The present invention solves shortcomings of conventional technology by applying an external surface electric field to achieve the objects of rapid and specific hybridization.

### SUMMARY OF THE INVENTION

One object of the present invention is to provide an apparatus for rapid hybridization reaction of nucleic acids, comprising a holder for loading a substrate and an electric field generator; wherein the substrate is detachable, as well as the nucleic acids and a buffer layer are disposed on the substrate; and said electric field generator comprises a power controller wherein at least one electrode is connected to the power controller and said electrode is separated from the substrate and is disposed above or below the holder; whereby a direction-convertible surface electric field is generated by the electric field generator.

Another object of the present invention is to provide a process for rapid hybridization reaction, comprising the following steps: (a) providing a substrate-immobilized nucleic acid sample in a holder, which is placed between a first electrode and a second electrode wherein the first electrode is at one end of the holder and the second electrode is at the opposite end of the holder; said substrate is detachable and separated from the electrodes; (b) adding a nucleic acid probe-containing solution to said substrate-immobilized nucleic acid of step (a); (c) applying a voltage to said first electrode for a period of time sufficient to permit hybridization of said probes to said substrate-immobilized nucleic acid; and (d) applying an opposite polarity voltage to said second electrode for a period of time sufficient to attract mismatched nucleic acid probes away from said substrate-immobilized nucleic acid.

### BRIEF DESCRIPTION OF THE DRAWINGS

FIG. 1 is a structure diagram of the apparatus according to the invention;

FIG. 2 is a sideview of the structure of the apparatus according to the invention;

FIG. 3 shows a substrate with nucleic acids adsorbed thereon;

FIG. 4a˜FIG. 4f depict rapid hybridization reaction steps according to the invention;

FIG. 4a depicts heating;

FIG. 4b depicts nucleic acids denatured from double strands to single strands after heating;

FIG. 4c depicts applying an upward electric field and probe nucleic acids moving downwards;

FIG. 4d depicts the electric field being zeroed;

FIG. 4e depicts applying a downward electric field and probe nucleic acids moving upwards;

FIG. 4f depicts applying an upward electric field and probe nucleic acids moving downwards;

FIG. 5 depicts another embodiment of the invention; and

FIG. 6 depicts another embodiment of the invention.

### DETAILED DESCRIPTION OF THE INVENTION

The invention relates apparatus and process for rapid hybridization which sustain reaction specificity and equal opportunity as well as speed up reaction rate for hybridization of nucleic acids.

One object of the invention is to provide an apparatus for rapid hybridization reaction of nucleic acids comprising:

a holder for loading a substrate; and

an electric field generator;

wherein the substrate is detachable, as well as the nucleic acids and a buffer layer are disposed on the substrate; and said electric field generator comprises a power controller wherein at least one electrode is connected to the power controller and said electrode is separated from the substrate and is disposed above or below the holder;

whereby a direction-convertible electric field is generated by the electric field generator.

According to the invention, the substrate is detachable and separated from the electrode. The substrate can be easily replaced after the completion of the hybridization reaction. Any suitable materials can be used to prepare the substrate. Preferably, the substrate is selected from silica, glass, a semiconductor, a nylon membrane, a nitrocellulose membrane or filter paper.

According to the invention, the electrode may be a flat plate or curved plate and disposed above or below the holder. In one preferred embodiment of the invention, the apparatus comprises two electrodes wherein one with positive charge is at the end of the holder side and another with negative charge is at the opposite end of the holder. The nucleic acids with negative charge can be attracted by said positive electrode and expelled by said negative electrode. The reaction rate of the nucleic acid hybridization can be thus increased.

According to the invention, the electric field generated by the electric field generator is a surface electric field. The surface electronic field can produce an evenly attractive power to the nucleic acid molecules. Such nucleic acid molecules can distribute evenly over the substrate. An uneven hybridization reaction can be avoided.

In one embodiment of the invention, the apparatus optionally further comprises a motor for rotating the holder to further accelerate the reaction rate. The motor can agitate the reaction solution to increase the collision rate of the reactants.

In another embodiment of the invention, the apparatus optionally further comprises a temperature controller for controlling the reaction temperature below the temperature of causing denature of the nucleic acid. In another further embodiment of the invention, the apparatus can further comprises the electrodes on both sides of the holder for generating an electric field in a horizontal direction.

Another object of the invention is to provide a process for rapid hybridization reaction, comprising the following steps:

(a) providing a substrate-immobilized nucleic acid sample in a holder, which is placed between a first electrode and a second electrode wherein the first electrode is at one end of the holder and the second electrode is at the opposite end of the holder; said substrate is detachable and separated from the electrodes;

(b) adding a nucleic acid probe-containing solution to said substrate-immobilized nucleic acid of step (a);

(c) applying a voltage to said first electrode for a period of time sufficient to permit hybridization of said probes to said substrate-immobilized nucleic acid; and

(d) applying an opposite polarity voltage to said second electrode for a period of time sufficient to attract mismatched nucleic acid probes away from said substrate-immobilized nucleic acid.

According to the invention, the process may further comprise a step of zeroing the electric field before the step (c). Also, the process optionally further comprises rotating the holder at the steps of (b) and (c) with a rotating motor to further accelerate the reaction rate, heating in steps (c) and (d) below the temperature of causing the denature of the nucleic acid; or applying an electric field in a horizontal direction; or heating. The steps (b) and (c) are alternatively repeating for at least 3 times.

### DESCRIPTION OF THE PREFERRED EMBODIMENTS

In one embodiment, the invention places a substrate with sample nucleic acid adsorbed thereon in the mid of two electrodes. The electrode below the substrate is positively charged and the electrode above is negatively charged. The objects of rapid hybridization reaction are achieved by setting the electric field since the nucleic acid; DNA or RNA is negatively charged and is attracted by a positive electrode and expelled by a negative electrode.

The preferred embodiment of the invention is exemplified by the following figures, whereas it should be noticed that the figures are exemplified for illustration and not for confining the scope of the invention.

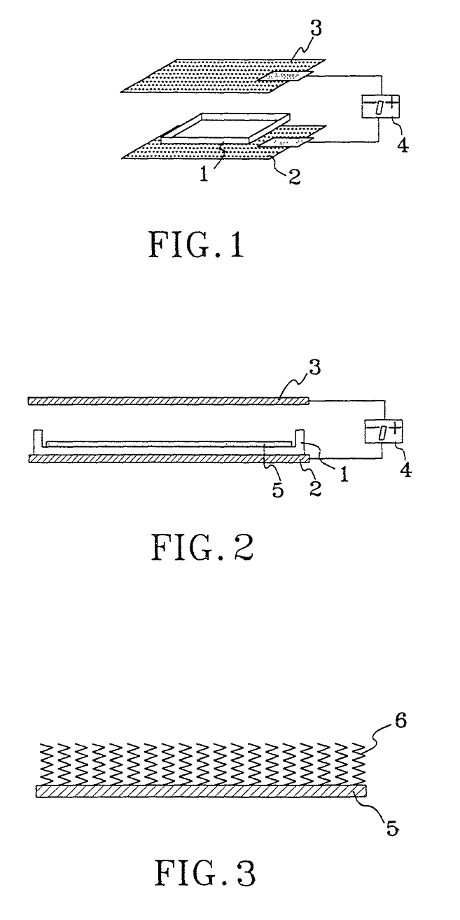

A substrate, hybridization substances and a buffer solution (not shown in the figure) are accepted in a holder 1 of the apparatus according to the invention as shown in FIG. 1. A lower electrode 2 is placed below and an upper electrode 3 above the holder 1. The electrodes are flat or curved plates made by any conductive materials and a potential is supplied to the electrodes by a power controller 4. The potential ranges from 10 volts to 10,000 volts. A direction-convertible electric field at least covering the holder and the substances is generated by the electrodes. FIG. 2 is a sideview of the structure of the apparatus in the preferred embodiment, wherein a substrate 5 in the holder 1 is specified. The substrate 5 with scaled-up sample nucleic acids 6 adsorbed thereon is shown in FIG. 3.

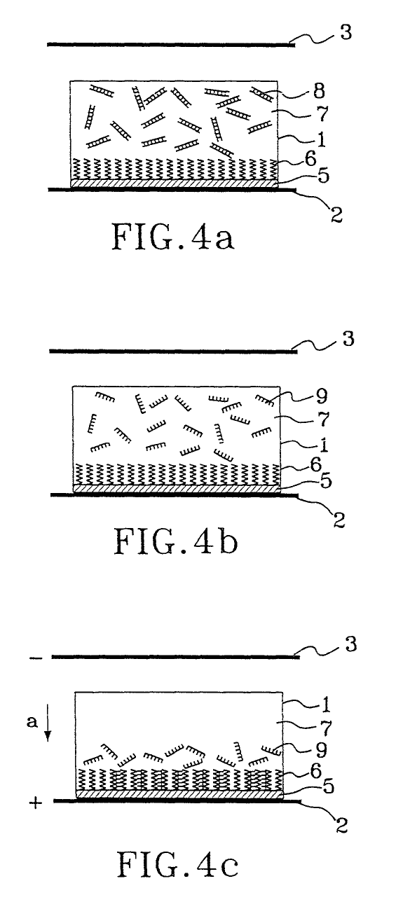

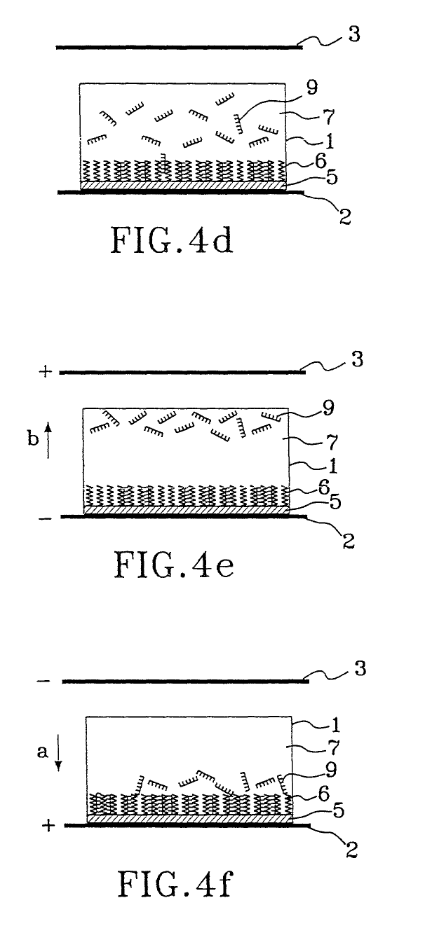

The steps of rapid hybridization reaction according to the invention are shown from FIG. 4a to FIG. 4f. A buffer layer 7 and probe nucleic acids (double strands) 8 in the holder 1 (not in actual proportion) are heated as shown in FIG. 4a. The heat denatures the probe nucleic acids into single stranded probe nucleic acids 9 as shown in FIG. 4b. Then upward electric field is applied to the probe nucleic acids as shown in FIG. 4c. The upward electric field is generated by the positive potential of the lower electrode 2 and the relatively negative potential of the upper electrode 3. The intrinsically negative probe nucleic acids 9 are attracted by the electric field and move in direction “a” rapidly towards the substrate 5. The effective duration of the upward electric field lasts from 5 seconds to 5 minutes, preferably from 30 seconds to 3 minutes.

All-around attraction forces are applied to the whole buffer layer since the electric field is generated by surface electrodes. Hybridization opportunity for each nucleic acid molecule is equal. The electric field is then zeroed as shown in FIG. 4d. The effective duration of the zero electric field lasts from 5 seconds to 5 minutes, preferably from 30 seconds to 3 minutes. The probe nucleic acid reacts with a complementary sample nucleic acid. However, the probe nucleic acid mismatches a non-complementary sample nucleic acid and results in pseudo-positive detection as the rapid movement of the probe nucleic acid towards the sample nucleic acid. At least one time of reversing the direction of the electric field is required to expel the mismatched pairs. As shown in FIG. 4e, by applying a downward electric field the unmatched and mismatched probe nucleic acid (single stranded) 9 moves upwards in direction “b” away from the substrate 5 and sample nucleic acid 6. The electric field is generated by the negative potential of the lower electrode 2 and relatively positive potential of the upper electrode 3. The effective duration of the downward electric field lasts from 5 seconds to 5 minutes, preferably from 30 seconds to 3 minutes. The converted electric field distinguishes matched nucleic acids from mismatched ones because pairs of highly-bonded complementary nucleic acids are not affected by the electric field and keep remaining on the substrate. The reaction time is greatly reduced, in the mean time, the specificity is retained.

In order to increase completeness of the hybridization reaction, the electric field may be converted repeatedly. An upward electric field is applied again as shown in FIG. 4f and probe nucleic acids including ones not reacting in time move downwards rapidly. The repeatedly direction-converted electric field not only increases mixing and hybridization opportunity but also allows probe nucleic acids not reacting in time to approach complementary sample nucleic acids again (when the electric field direction is upward) and separates mismatched probe nucleic acids from sample nucleic acids which are not completely complementary (when the electric field direction is downward). The precision is greatly enhanced.

The hybridization reaction could be finished in several minutes according to the invention compared to the more than ten hours for conventional art. The reaction time is reduced by a factor from 60 to 100.

Example: controlling a hybridization reaction by a surface electric field

Sample DNA was heated at 95°C. for 5 minutes and denatured from double strands to single ones. After being placed on ice for one or two minutes, the sample DNA was adsorbed on 4 cm2 (2 cm×2 cm) nylon membranes (positively charged, Boehringer Manngeim Biochemicals BM; cat. no. 1 209 299) and fixed thereon by drying at 80°C. for two hours.

The nylon membranes dotted with nucleic acids were placed in a plastic holder (L×W×H: 5.5 cm×4 cm×0.5 cm) and then placed in the mid of two electrodes which were spaced 2 cm apart (each L×W×H: 10 cm×10 cm×0.5 cm). One milliliter of a hybridization solution (5×SSC; 0.1% (w/v) N-lauroylsarcosine; 10% (w/v) SDS; 1× blocking buffer, Boehringer Manngeim Biochemicals BM; cat. no. 1093 657, DIG DNA Labeling and Detection Kit) [20 ml/100 cm2] was added into the holder and preheated at 68°C. for 30 minutes.

Dissolving probe nucleic acids labeled with DIG in another hybridization solution and the probe nucleic acids were denatured by heating at 95°C. for 5 minutes (5-25 ng/ml, ml)[2.5 ml/100 cm2]. After being placed on ice for one or two minutes, the probe nucleic acids were added to the nylon membranes. At 42°C., the electrode below the holder was positively charged on 60 volts and the one above the holder was negatively charged on minus 60 volts. The hybridization reaction lasted one minute and then the electric field was zeroed for 30 seconds. The potential of the electrodes was reversed as 10 volts on the upper electrode and minus 10 volts on the lower electrode. The hybridization lasted 30 seconds and then the electric field was zeroed for 30 seconds again. The conversion of electric charge repeated for ten times in order to avoid mismatched hybridization.

The substrate was rinsed at room temperature for five minutes by 2× SSC of 0.1% SDS and then anti-DIG antigen labeled with alkaline phosphatase [2 μl anti-DIG-AP conjugate/20 ml 1×blocking solution] was added. After 30 minutes at room temperature, the substrate was rinsed twice with a maleic acid buffer (0.1M maleic acid, 0.15M NaCl, pH7.5) at room temperature, each rinse lasted 15 minutes. The nylon membranes were immersed in a detection buffer (0.1M Tris-HCl; 0.1M NaCl; 50 mM MgCl2, pH9.5) for 5 minutes and then mixed with a fresh color-substrate solution (45 μl NBT solution mixed with 35 μl X-phosphate solution and added a detection buffer to 10 ml) in the dark for 10 minutes. Finally the membranes were rinsed with an onefold TE buffer solution to stop reaction and air-dried at room temperature.

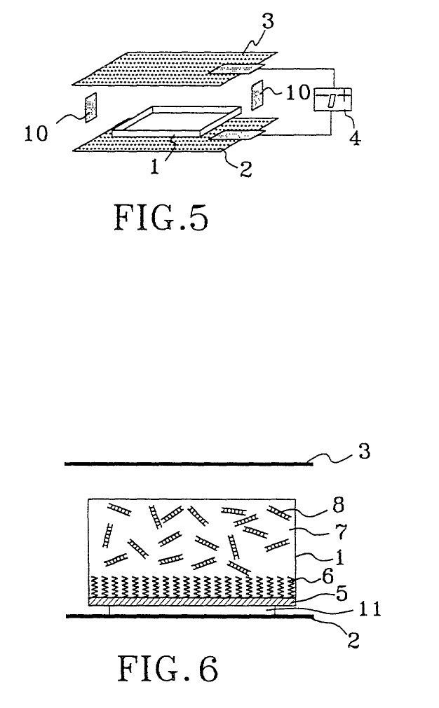

In one embodiment of the invention, electrodes 10 are placed face to face in a horizontal direction as shown in FIG. 5; and in another embodiment a rotator 11 is placed below the holder 1 as shown in FIG. 6 in order to enhance mixing.

With the disclosed invention, apparently numerous modifications and variations can be made without departing from the scope and spirit of the present invention. Therefore the present invention is intended to be limited only as indicated in the following claims.

What is claimed is:

1. An Apparatus for rapid hybridization reaction of nucleic acids, comprising:
a holder for loading a substrate; and
an electric field generator;
wherein the substrate is detachable, as well as the nucleic acids and a buffer layer are disposed on the substrate; and said electric field generator comprises a power controller wherein at least one electrode is connected to the power controller and said electrode is separated from the substrate and is disposed above or below the holder; whereby a direction-convertible electric field is generated by the electric field generator.

2. The apparatus for rapid hybridization reaction of claim 1, wherein said electric field generated by the electric field generator is a surface electric field.

3. The apparatus for rapid hybridization reaction of claim 1, wherein said electrode is a flat plate.

4. The apparatus for rapid hybridization reaction of claim 1, wherein said electrode is a curved plate.

5. The apparatus for rapid hybridization reaction of claim 1, which can comprise two electrodes wherein one with positive charge is at the end of the holder side and another with negative charge is at the opposite end of the holder.

6. The apparatus for rapid hybridization reaction of claim 1, wherein said substrate is silica.

7. The apparatus for rapid hybridization reaction of claim 1, wherein said substrate is glass.

8. The apparatus for rapid hybridization reaction of claim 1, wherein said substrate is a semiconductor.

9. The apparatus for rapid hybridization reaction of claim 1, wherein said substrate is a nylon membrane.

10. The apparatus for rapid hybridization reaction of claim 1, wherein said substrate is a nitrocellulose membrane.

11. The apparatus for rapid hybridization reaction of claim 1, wherein said substrate is filter paper.

12. The apparatus for rapid hybridization reaction of claim 1 further comprising a motor for rotating said holder to further accelerate the reaction rate.

13. The apparatus for rapid hybridization reaction of claim 1 further comprising a temperature controller for controlling the reaction temperature below the temperature of causing the denature of the nucleic acid.

14. The apparatus for rapid hybridization reaction of claim 1 further comprising electrodes on both sides of the holder for generating an electric field in a horizontal direction.

15. A process for rapid hybridization of nucleic acids comprising the following steps:
(a) providing a substrate-immobilized nucleic acid sample in a holder, which is placed between a first electrode and a second electrode wherein the first electrode is at one end of the holder and the second electrode is at the opposite end of the holder; said substrate is detachable and separated from the electrodes;
(b) adding a nucleic acid probe-containing solution to said substrate-immobilized nucleic acid of step (a);
(c) applying a voltage to said first electrode for a period of time sufficient to permit hybridization of said probes to said substrate-immobilized nucleic acid; and
(d) applying an opposite polarity voltage to said second electrode for a period of time sufficient to attract mismatched nucleic acid probes away from said substrate-immobilized nucleic acid.

16. The process for rapid hybridization reaction of claim 15, further comprising a step of zeroing the electric field before the step (c).

17. The process for rapid hybridization reaction of claim 16, further comprising rotating the holder at the steps of (b) and (c) with a rotating motor to further accelerate the reaction rate.

18. The process for rapid hybridization reaction of claim 15, further comprising heating in steps (c) and (d) below the temperature of causing the denature of the nucleic acid.

19. The process for rapid hybridization reaction of claim 15, wherein the steps (b) and (c) are alternatively repeating for at least 3 times.
